(19) United States
(12) Patent Application Publication
(10) Pub. No.: US 2012/0187041 A1
(43) Pub. Date: Jul. 26, 2012

(54) INSECT-BASED REMOVAL OF ORGANIC SOLUTES FROM LIQUID

(75) Inventors: Radu Popa, Portland, OR (US); Terrence R. Green, Lake Oswego, OR (US)

(73) Assignees: Portland State University; The State of Oregon Acting By and Through the State Board of Higher Education on Behalf of

(21) Appl. No.: 13/010,654

(22) Filed: Jan. 20, 2011

(51) Int. Cl. C02F 3/32; C02F 1/58; A01K 67/00

(52) U.S. Cl. 210/602; 119/6.5; 210/198.1; 210/85; 210/177; 210/188

(57) ABSTRACT

Disclosed herein are methods of removing organic solutes from liquids. The methods include incubating fly larvae with a liquid that contains organic solutes. Also disclosed herein are methods of producing larva biomass. Additionally, apparatus for use with the disclosed methods are described.

### FIELD

The disclosure relates generally to a process for removing organic solutes from a liquid by incubating fly larvae with a liquid containing organic solutes, and apparatus designed for such processing of liquids. Certain embodiments relate to increasing larval biomass.

### BACKGROUND

Composting technologies for processing organic material, including animal manure, sewage, agricultural and forest residues, industrial byproducts, produce and food scrap are well-known. For example, microbial-dependent aerobic and anaerobic composting, vermicomposting and processes combining microbial and vermicomposting technologies are useful to dissimilate organic material. However, technologies concerning detoxification and treatment of liquids containing organic solutes, including liquid produced by the use of known composting technologies, are underdeveloped. During these processes numerous byproducts are released, including gases (CO₂, CH₄, N₂O, NH₃, NO, H₂S, methyl sulfides), alcohols (ethanol and methanol), volatile organic acids (VOAs, acetic, propionic, butyric, valeric and isovaleric), amines, mercaptans, sugars, proteins and many other biochemicals. Many of these compounds are also present in the organic material before composting.

Fly larvae will feed on a variety of organic materials, are known to be useful as a manure management tool and they have economic value as a feedstock (Newton et al., J. Anim. Sci., 44:395-400, 1977; Bondari and Sheppard, Aquaculture and Fisheries Management, 18:209-220, 1987; Sheppard et al., Bioresource Technology, 50:275-279, 1994; Tomberlin et al., Ann. Entomol. Soc. Am., 95:379-386, 2002; St-Hilaire et al., J. World Aquaculture Society, 38:59-67, 2007; St-Hilaire et al., J. World Aquaculture Society, 38:309-313, 2007). One example is Black Soldier Fly (BSF) larvae. However, known methods of using BSF larvae for processing of organic material call for the use of organic material with a moisture content of less than ˜80% water by weight (Fatchurochim et al., J. Entomol. Sci., 24:224-231, 1989).

### SUMMARY

Disclosed herein is the unexpected discovery that larvae (e.g., fly larvae) can consume and assimilate organic solutes present in a liquid and gain biomass based on this nutrient source. Thus, surprisingly, larvae can be used to remove organic solutes from a liquid and incubating larvae with liquid containing organic solutes will cause the larvae to gain biomass. An apparatus for performing these processes is disclosed herein.

A method of removing organic solute from a liquid is provided. The method comprises selecting a liquid nutrient source containing organic solute, wherein the nutrient source comprises at least 80% water or other liquid and is substantially free of solid nutrients; and incubating fly larvae for a period of time with the nutrient source, thereby removing organic solute from the liquid. In some embodiments, the method further comprises harvesting the larvae.

Also provided is a method of producing larva biomass, comprising incubating fly larvae for a period of time with a liquid nutrient source containing organic solute, wherein the nutrient source comprises at least 80% water or other liquid and is substantially free of solid nutrients; and harvesting the fly larvae, thereby producing larva biomass.

In some embodiments, the fly larvae used in the methods provided herein are Hermetia illucens larvae.

Also provided is an apparatus for removing organic solute from a liquid or for producing larva biomass, the apparatus comprising an enclosed tank, which enclosed tank comprises a liquid entry port, a liquid exit port, a gas entry port, a gas exit port, an inner reservoir capable of holding liquid, wherein the liquid entry and exit ports are operably linked to the inner reservoir, and access means for accessing the inner reservoir of the tank. In some embodiments, the inner reservoir contains one or more fly larvae, such as Hermetia illucens (black soldier fly; BSF) larvae.

The foregoing and other aspects, objects, features, and advantages of the invention will become more apparent from the following detailed description, which proceeds with reference to the accompanying figures.

### BRIEF DESCRIPTION OF THE FIGURES

FIG. 1 illustrates an embodiment of the disclosed apparatus for removing organic solute from a liquid nutrient source or increasing larva biomass.

FIG. 2 shows that BSF-larvae clear short chain alcohols and VOAs from compost tea. Short chain alcohol and VOA concentrations in control and BSF-larvae treated compost tea were measured by gas chromatography. Samples were drawn from each condition at the beginning of the experiment (“T₀”), and two days later (“2 day tea”), and test outcomes compared. Concentrations of ethanol (column set 1), propanol (column set 2), acetic acid (column set 3), propanoic acid (column set 4), butyric acid (column set 5) and isovaleric acid (column set 6) were measured. These results show that BSF feeding on compost tea can clear short chain alcohols and VOAs from the liquid nutrient source.

FIG. 3 shows that BSF-larvae clear ninhydrin-positive amines from compost tea. Ninhydrin-positive amines in control and BSF-larvae treated compost tea were analyzed by thin layer chromatography. Samples were applied as follows (left to right): lane 1, glycine; lane 2, glycyl-glycine; lane 3, glutamic acid; lane 4, compost tea control; lane 5, compost tea processed with BSF larvae; lane 6, leucine. The results of this experiment show that BSF larvae can clear nitrogen metabolites from compost tea.

FIG. 4 shows the growth of BSF larvae (% weight gain) over a one week interval while they fed on various filtered liquid nutrient sources, including Urine (Ur), Sewage water (SW), Water extracted Gainesville House Fly diet (GF), Chicken broth (CB), Orange juice (OJ), Compost tea (CT), Chicken manure extract (CME), Whey (Wh); and Milk (Mk).

FIG. 5 shows an image of a Modular Larvae Incubation Unit (MLIU), showing drain holes, used to incubate BSF larvae with circulating fluids as described in Example 5.

FIG. 6 shows a side view of an operating array of MLIUs housed inside an enclosed tank as described in Example 5.

FIG. 7 shows a top down view of the uppermost MLIU in an array of MLIUs as described in Example 5. The BSF larvae are incubating in compost tea which enters the array of MLIUs from a liquid entry port and drips through the holes of the uppermost MLIU into the next MLIU in the array.

FIG. 8 shows the bottom MLIU, which lack drainage holes, for use in an array of MLIUs, as described in Example 5. A tube for draining liquid nutrient source from the bottom MLIU is shown.

FIG. 9 shows an assembled apparatus as described in Example 5, and for use with the methods described herein. The apparatus including an enclosed tank having an inner reservoir comprised of a single array of MLIUs. Each of the MLIUs (except the bottom MLIU) contains BSF larvae which are feeding on compost tea pumped through the apparatus.

### DETAILED DESCRIPTION

### I. Terms and Abbreviations

BSF black soldier fly

MLIU modular larvae incubation unit

VOA volatile organic acid

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a Black Soldier Fly larva” includes single or plural Black Soldier Fly larvae and is considered equivalent to the phrase “comprising at least one Black Soldier Fly larva.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. For example, conventional methods well known in the art to which a disclosed invention pertains are described in various general and more specific references, including, for example, Smith and March, March's Advanced Organic Chemistry: Reactions, Mechanisms, and Structure, Fifth Edition, Wiley-Interscience, 2001; Vogel, A Textbook of Practical Organic Chemistry, Including Qualitative Organic Analysis, Fourth Edition, New York: Longman, 1978; and International Union of Pure and Applied Chemistry, Nomenclature of Organic Chemistry, Sections A, B, C, D, E, F, and H, Oxford: Pergamon, 1979. Additional terms commonly used in chemistry can also be found in these references.

Biomass: A mass of organic material formed by a living organism. Larva biomass is a mass of fly larvae.

Compost: The decomposed or decomposing remnants of organic material, such as plant materials, food scraps, or animal feces and urine.

Composting: The process of decomposition that allows organic material to decompose into compost. Many types of composting are known, including aerobic, anaerobic and insect-based composting.

Compost tea: A liquid produced by separating liquid from a compost/liquid mixture. For example, compost tea can be produced by adding a liquid, such as water, to compost to make a compost/liquid mixture, followed by separating the liquid from the mixture. Compost tea can also be produced by separating liquid from organic matter having a liquid content; for example, by separating liquid from a mixture comprising animal feces and urine.

Control: Samples believed to be normal (e.g., representative of an activity or function in the absence of the variable being tested), as well as laboratory values, even though possibly arbitrarily set, keeping in mind that such values can vary from laboratory to laboratory. A control group is practically identical to the treatment group, except for the single variable of interest whose effect is being tested, which is only applied to the treatment group.

Contacting: Placement in direct physical association, including in solid, liquid and gas form. Contacting includes contact between one molecule and another molecule and also includes contact between a larva and a liquid nutrient source.

Fly larvae: Also known as maggots, fly larvae are a stage of the life cycle of a fly, which progresses through (in order) egg, larva, pupa and adult fly stages. Several intermediate stages are also identified, including the pre-pupa stage, a stage in between the larva and pupa stages when the larva moves away from the nutrient source to find a pupation site. Examples of fly larvae include Musca domestica larvae, Muscina stabulans larvae, Fannia canicularis larvae, Fannia femoralis larvae, Ophyra aenescens larvae, or Hermetia illucens larvae. As used herein, “larvae” includes fly larvae, such as Hermetia illucens and other fly larvae.

Enclosed tank: An enclosed container made of a substance that is substantially impermeable to air. Such a container may have openings designed to allow liquid, solid or gas to enter and/or exit the container, for example liquid entry and exit ports, or gas entry and exit ports. The container may be of any size. In some examples, the container has a lid or door that opens to allow access to the interior of the container.

Gas scrubber: A device that can be used to remove particulates and/or gases from air. Gas scrubbers include scrubbers designed for wet scrubbing and dry scrubbing.

Harvesting: The collection of something, by any means. For example, harvesting larvae includes collecting larvae by hand or by machine, among other methods.

Hermetia illucens: Commonly known as the Black Soldier Fly or Privy Fly, Hermetia illucens is a fly of the family Stratiomyidae.

Incubating: A term that includes a sufficient amount of time for a larva, such as a BSF larva, to interact with something, such as a liquid nutrient source.

Liquid nutrient source: A liquid containing organic solutes. A liquid nutrient source is substantially free of solid nutrients. A liquid nutrient source is at least about 80, 81, 82, 83, 84, 85, 86, 87, 88, 89, 90, 91, 92, 93, 94, 95, 96, 97, 98, 99 percent liquid by weight. Though some or all of the liquid portion of a “liquid nutrient source” can be water, water is not the only liquid that is contemplated. Other non-limiting examples of liquids that may be components of a “liquid nutrient source” include alcohols and other organic solvents. A liquid has the property of dripping and flowing, and is capable of being pumped through a pipe. Non-limiting examples of liquid nutrient sources include whey, compost tea, liquid produced from sewage, grey water, liquid produced from food processing and combinations of two or more thereof. A liquid nutrient source may contain microorganisms, for example aerobic and anaerobic bacteria. A liquid nutrient source stream is a liquid nutrient source that is flowing.

Manure: Material, especially barnyard or stable dung, often with discarded animal bedding, used to fertilize soil. For example, manure includes organic material excreted by animals feeding on varying feeds, which may be mixed with feces and urine, compost and plant material.

Modular Larvae Incubation Unit (MLIU): A partially or fully enclosed container made of a substance that is substantially impermeable to liquid, e.g., metal, rubber, wood or plastic, which is used for incubating larvae with a liquid nutrient source. Some MLIUs comprise means for liquid to pass through the bottom of the MLIU, for example, an MLIU may comprise holes in the bottom surface of the MLIU.

In some embodiments, the MLIUs are included in an array of MLIUs, wherein the MLIUs are positioned, such that at least one MLIU is above at least one other MLIU. In some embodiments, the uppermost module in an array of MLIUs includes a lid. Typically, each MLIU in an array (except the lowest unit in an array) has means for liquid nutrient source to pass through its lower surface. The lowest MLIU in an array of MLIUs usually lacks drain holes, allowing liquid nutrient source traveling through the array of MLIUs to collect at least temporarily in the lowest unit. Any number of MLIUs may be included in an array of MLIUs. In some cases, multiple MLIUs will be located above one or more MLIUs. In each array of MLIUs, liquid nutrient source may pass from higher MLIUs to lower MLIUs. An array of MLIUs may include MLIUs stacked directly above one another, or may include MLIUs stacked partially above one another, or a combination thereof. Some MLIUs are constructed to be capable of connecting to other MLIUs, others are not capable of connecting to other MLIUs. For example, an array of MLIUs may include MLIUs capable of interconnecting vertically to form vertical arrays of MLIUs, e.g., by snapping together snuggly one on top of the other in vertical stacks of repeating units.

Organic compound: A gaseous, liquid, or solid chemical compound, the molecules of which contain carbon, nitrogen, sulfur, phosphorous or a combination of two or more thereof. Various organic compounds are listed in International Union of Pure and Applied Chemistry, Nomenclature of Organic Chemistry, Sections A, B, C, D, E, F, and H, Oxford: Pergamon, 1979. Examples include gases (CO₂, CH₄, N₂O, NH₃, NO, H₂S, methyl sulfides), alcohols (ethanol and methanol), volatile organic acids (VOAs, acetic, propionic, butyric, valeric and isovaleric), amines, mercaptans, sugars, proteins and many other biochemicals.

Organic material: Solid and/or liquid matter composed of organic compounds.

Organic solute: An organic compound dissolved in fluid, forming a solution.

Sewage: Water-based fluid containing organic matter and solutes. Sewage may include feces and urine from human and non-human animals. Sewage may include waste from human activities, for example, blackwater (e.g., toilet and dishwasher waste) and grey water (e.g. waste water generated from washing activities). Residential, institutional, commercial and industrial establishments may produce sewage, including waste from toilets, baths, showers, kitchens, sinks, etc. Typically, sewage is waste intended to be carried away from the source of the waste, for example, carried to a sewage treatment facility.

Vermicompost: The product of composting utilizing various species of worms, usually to create a heterogeneous mixture of decomposing vegetable or food waste, bedding materials, and vermicast. Vermicast, also known as worm castings, worm humus or worm manure, is the end-product of the breakdown of organic matter by species of earthworms. The earthworm most often used is the Red Wiggler (Eisenia foetida or Eisenia andrei). Containing water-soluble nutrients, vermicompost is a nutrient-rich organic fertilizer and soil conditioner. Methods of using worms to produce vermicompost are well known (see, e.g., U.S. Pat. Nos. 6,223,687, 6,838,082, 6,890,438, 7,141,169).

### II. Overview of Several Embodiments

Provided here in are methods of reducing and/or removing organic solutes from a liquid and methods of increasing larval biomass, as well as apparatuses and devices for performing these methods.

A method of removing organic solute from a liquid is provided herein. The method comprises selecting a liquid nutrient source containing organic solute, wherein the nutrient source comprises at least 80% water or other liquid and is substantially free of solid nutrients; and incubating fly larvae for a period of time with the nutrient source, thereby removing organic solute from the liquid. In some embodiments, the method further comprises harvesting the larvae.

Also provided herein is a method of producing larva biomass, comprising incubating fly larvae for a period of time with a liquid nutrient source containing organic solute, wherein the nutrient source comprises at least 80% water or other liquid and is substantially free of solid nutrients; and harvesting the fly larvae, thereby producing larva biomass.

In some embodiments, the liquid nutrient source comprises compost tea, liquid produced from urine, whey, sewage, liquid produced from manure, or a combination of two or more thereof. In some embodiments, the pH of the liquid nutrient source is about 3.2 to about 9.4.

In some embodiments, the fly larvae used in the methods provided herein are selected from the group consisting of Musca domestica larvae, Muscina stabulans larvae, Fannia canicularis larvae, Fannia femoralis larvae, Ophyra aenescens larvae, Hermetia illucens larvae or a combination of two or more thereof.

In some embodiments, the fly larvae are incubated at about 2×10³ to about 2×10⁵ larvae per liter of liquid nutrient source, at a temperature of about 20° C. to about 40° C., with the liquid nutrient source for about 1, 6, 12, 24 or 48 hours, or about 1 day, 1 week or 1 month, or a combination of two or more thereof.

In some embodiments, harvesting the larvae comprises harvesting mature larvae or larvae entering the pupa stage of the larva life cycle, or both. In various embodiments, harvesting the larvae comprises passing the larvae through a filter having a pore size of about 0.3 cm to about 0.5 cm.

Also provided herein is an apparatus for removing organic solute from a liquid or for producing larva biomass. In some embodiments, the apparatus comprises an enclosed tank, which enclosed tank comprises a liquid entry port, a liquid exit port, a gas entry port, a gas exit port, an inner reservoir capable of holding liquid, wherein the liquid entry and exit ports are operably linked to the inner reservoir, and access means for accessing the inner reservoir of the tank. In some embodiments, the inner reservoir contains one or more fly larvae, such as Hermetia illucens (black soldier fly; BSF) larvae. In some embodiments, the inner reservoir comprises one or more arrays of modular larvae incubation units.

In some embodiments of the apparatus, the gas exit port is operably linked to a gas pump. Optionally, the apparatus is operably linked to at least one gas trapping scrubber means.

In some embodiments, the apparatus further comprises means for maintaining fly larva at a depth of about 0.5 to about 3 cm of liquid in the inner reservoir, means for preventing fly larvae from passing through the liquid exit port, means for removing larvae from the inner reservoir, means to monitor organic solute concentration of the liquid in the liquid entry port, the inner reservoir, the liquid exit port or a combination of two of more thereof, means for heating or cooling a liquid in the inner reservoir, or a combination of two or more thereof.

It will be further understood that the methods of reducing organic solute in a liquid or producing larva biomass, as well as the apparatus for performing these methods disclosed herein are useful beyond the specific circumstances that are described in detail herein, and for instance are expected to be useful for any number of situations where it is desirable to remove organic solute from liquid or increase insect larva biomass.

### III. Larvae

The larva growth stage is a stage of the life cycle of a fly, which progresses from egg to larva to pupa to adult fly stages. Several intermediate stages are also identified, including the pre-pupa stage, a stage in between the larva and pupa stages when the larva moves away from the nutrient source to find a pupation site. The skilled artisan is familiar with larvae generally, and with methods of breeding and propagating larvae. For example, methods of breeding and propagating Musca domestica larvae, Muscina stabulans larvae, Fannia canicularis larvae, Fannia femoralis larvae, Ophyra aenescens larvae, or Hermetia illucens larvae (see, e.g., Fatchurochim et al., J. Entomol. Sci., 24:224-231, 1989).

Some embodiments use BSF larvae. BSF larvae feed on a variety of vegetal and manure wastes of varying extreme pH ranges and O₂ tensions, self-harvest on entering the pupae stage from the organic matter they are feeding on, and are ubiquitous throughout much of the world extending between roughly the equator and 45^{th} degree latitude (Newton et al., J. Anim. Sci., 44:395-400, 1977; Bondari and Sheppard, Aquaculture and Fisheries Management, 18:209-220, 1987; Sheppard et al., Bioresource Technology, 50:275-279, 1994; Tomberlin et al., Ann. Entomol. Soc. Am., 95:379-386, 2002; St-Hilaire et al., J. World Aquaculture Society, 38:59-67, 2007; St-Hilaire et al., J. World Aquaculture Society, 38:309-313, 2007). Adult BSF do not need to eat; they survive on the fat stored from the larva stage. BSF larva consume organic matter, including kitchen waste, spoiled feed, and manure, and assimilates organic compounds in the organic matter into larva biomass.

Methods of breeding and propagating BSF larvae, including methods of breeding BSF larvae in captivity, as well as methods of using BSF larvae to process solid wastes, are familiar to the skilled artisan (see for example, Tomberlin et al., Environ Entomol., 38 (3):930-4, 2009; Sheppard et al., J. Med. Entomol., 39 (4):695-8, 2002; Tomberlin, J. Econ. Entomol., 95:598-602, 2002; U.S. Pat. No. 6,780,637). Additionally, BSF larvae can be purchased commercially, for example BioGrubs™ BSF larvae (Prota Culture, LLC, Dallas, Tex.) and Phoenix Worms™ BSF larvae (Insect Science Resource, LLC, Tifton, Ga.). Alternatively, BSF larvae, and eggs laid by adults, can be harvested in the wild by gathering eggs and larvae present in animal manure, particularly chicken and pig manure, on farms and at commercial animal facilities open to the elements, especially in warmer climates where the insects are known to lay eggs throughout the year in the wild.

BSF eggs take approximately 4 days to hatch and are typically deposited in crevices or on surfaces above or adjacent to the food source. BSF larvae approaching the pupae stage reach a size in excess of 2 cm in length and 0.4 cm in diameter relative to immature larvae which start out on hatching from eggs at less than 0.2 cm in length and less than 0.1 cm in diameter. Although they can be stored at room temperature for several weeks, their longest shelf life is achieved at 50-60° F. (10-16 C).

### IV. Liquid Nutrient Sources

Embodiments described herein utilize liquid nutrient sources. The liquid nutrient sources are substantially free of solid nutrients (such as solid organic matter). However, particulates of solid nutrients may be included in a liquid nutrient source. In the embodiments described herein, the liquid nutrient source is at least about 80, 81, 82, 83, 84, 85, 86, 87, 88, 89, 90, 91, 92, 93, 94, 95, 96, 97, 98, or 99 percent liquid by weight.

The liquid nutrient sources disclosed herein comprise liquids containing organic solute, e.g., dissolved organic compounds, including, gases (e.g., CO₂, CH₄, N₂O, NH₃, NO, H₂S, methyl sulfides), alcohols (e.g., ethanol and methanol), volatile organic acids (e.g., acetic, propionic, butyric, valeric and isovaleric), amines, mercaptans, sugars, proteins and many other biochemicals. Liquids containing organic solutes will be apparent to the skilled artisan. Examples include liquid produced from food processing (e.g., whey), liquid industrial waste or liquid produced from industrial waste, sewage (e.g., toilet waste), liquid produced from sewage and sewage treatment facilities, liquid produced from manure and compost tea. In some embodiments, the liquid nutrient source may contain microorganisms, for example, aerobic and anaerobic bacteria.

In some embodiments, the liquid comprises sewage or a liquid produced from sewage. The skilled artisan understands methods used to identify and obtain sewage and liquid produced from sewage. For example, sewage includes both blackwater (toilet and dishwasher waste) and grey water (wastewater that does not include human waste, e.g. waste water generated from washing activities). Residential, institutional, commercial and industrial establishments produce sewage, including waste liquid from toilets, baths, showers, kitchens, sinks and so forth. Liquid produced from sewage is typically obtained by filtering or separating sewage into solid and liquid phase (e.g., by physical treatment of sewage at a wastewater treatment facility).

Though some or all of the liquid portion of a liquid nutrient source can be water, water is not the only liquid that is contemplated. Other non-limiting examples of liquids that may be components of a liquid nutrient source include alcohols and other organic solvents.

The liquid nutrient sources disclosed herein may range in pH. The liquid nutrient source may be any pH in which a larva is capable of surviving for a period of time sufficient to gain biomass or remove organic solute from the liquid nutrient source. For example, the liquid nutrient source may have a pH of about 3.0 to about 10, including about 3.2 to about 9.4, or higher or lower pH. In some embodiments, the liquid nutrient source has a pH of about 3.0, 3.5, 4.0, 4.5, 5.0, 5.5, 6.0, 6.5, 7.0, 7.5, 8.0, 8.5, 9.0, 9.5, or about 10.0. In some embodiments the pH of the liquid nutrient source is not uniform throughout the liquid nutrient source, or the pH of the liquid nutrient source may change over the time that the larvae are incubated in the liquid nutrient source. The pH of a liquid nutrient source may be altered by adding acid or base to the liquid nutrient source.

The liquid nutrient sources disclosed herein may range in temperature. The liquid nutrient source may be any temperature in which a larva is capable of surviving for a period of time sufficient to gain biomass or remove organic solute from the liquid nutrient source. For example, the liquid nutrient source may have a temperature of about 20° C. to about 40° C. or higher or lower temperatures. In some embodiments, the liquid nutrient source has a temperature of about 20, 21, 22, 23, 24, 25, 26, 27, 28, 29, 20, 31, 32, 33, 34, 35, 36, 37, 38, 39, or 40° C. In some embodiments the temperature of the liquid nutrient source is not uniform throughout the liquid nutrient source, or the temperature of the liquid nutrient source may change over the time that the larvae are incubated in the liquid nutrient source. The temperature of the liquid nutrient source may be altered by cooling or heating.

Selecting a liquid nutrient source that may be useful in one of the provided methods usually involves identifying a liquid containing organic solute and selecting the liquid, thereby selecting a liquid nutrient source. Methods of identifying that a liquid contains organic solute are known to the skilled artisan, and representative examples are described herein. For example, a sample of a liquid may be obtained and tested for organic solute. If the sample contains organic solute, then the liquid is a liquid nutrient source and selecting the liquid is selecting a liquid nutrient source. In other examples, it will be apparent to the skilled artisan that a liquid contains organic solute, and is therefore a liquid nutrient source. For example, certain liquids (e.g., whey, compost tea, sewage, liquid produced from food processing) are known to contain organic solute. Selecting a liquid known to contain organic solute is a means of selecting a liquid nutrient source.

### V. Methods for Removing Organic Solutes and Producing Larva Biomass

Disclosed herein are methods of producing larva biomass as well as methods of removing organic solute from a liquid. In some embodiments, the method of producing larva biomass includes incubating larvae for a period of time with a liquid nutrient source containing organic solute, wherein the liquid nutrient source comprises at least about 80% water and is substantially free of solid nutrients and harvesting the larvae, thereby producing larva biomass. In other embodiments, the method of removing organic solute from a liquid includes selecting a liquid nutrient source containing organic solute, wherein the nutrient source comprises at least about 80% water and is substantially free of solid nutrients; and incubating larvae for a period of time with the nutrient source, thereby removing organic solute from a liquid.

Incubation with a Liquid Nutrient Source for a Period of Time

The embodiments described herein involve incubating larvae with a liquid nutrient source, for example incubating BSF larvae with compost tea. Incubation of larvae with a liquid nutrient source involves contacting a liquid nutrient source with one or more larvae. The skilled artisan will understand methods of contacting a larva with a liquid nutrient source.

In the embodiments described herein, the depth of the liquid nutrient source that the larvae are exposed to is limited. For example the depth of the liquid nutrient source that the larvae are exposed to may be altered by changing the depth of the liquid nutrient source, or by adjusting the depth that the larvae may descend into the liquid nutrient source, e.g., by placing a barrier in the liquid nutrient source, below which the larvae cannot descend. In the embodiments described herein, the maximum depth of the liquid nutrient source that the larvae are exposed to is about 0.5, 1, 2, 3, 4 or 5 cm. In some embodiments, the maximum depth of the liquid nutrient source that the larvae are exposed to is about 3 cm. In some embodiments, the maximum depth of the liquid nutrient source that the larvae are exposed to varies between about 0 and 5 cm, 0 and 3 cm, 0.5 and 5 cm, or 0.5 and 3 cm deep.

Incubating larvae with a liquid nutrient source includes incubating various densities of larvae with the liquid nutrient source. In some embodiments, the density of the larvae in the liquid nutrient source is a minimum of about 2×10³ larvae per liter of liquid nutrient source. In some embodiments, the density of the larvae in the liquid nutrient source is a maximum of about 2×10⁵ larvae per liter of liquid nutrient source. In other embodiments, the density of the larvae in the liquid nutrient source ranges from about 2×10³ to about 2×10⁵ larvae per liter of liquid nutrient source.

The period of time that larvae are incubated with a liquid nutrient source may be determined based on numerous factors, for example, the particular liquid nutrient source the larvae are incubated with, the consumption rate of organic solute by the larvae, or the growth stage of the larvae. Once a particular determining factor is reached, the larvae may be removed from the liquid nutrient source. Methods of removing larvae from a liquid nutrient source are known to the skilled artisan and further described herein, for example as described for methods of harvesting larvae. Additionally, mixed populations of larvae of varying growth stage and size may be used. In many examples, the period of time that larvae are incubated with a liquid nutrient source will be apparent to the skilled artisan based on previous incubation of particular larvae species with particular types of liquid nutrient sources.

In some embodiments, the larvae are incubated with the liquid nutrient source for about 1, 2, 3, 4, 5, 6, 7, 8, 9, 10, 12, 24, 36 or 48 hours, or about 1 day, 1 week, 2 weeks, 3 weeks, 1 month, time periods in between these time, or even more or less time. Optionally, the amount of time the larvae are incubated with the liquid nutrient source may be conveniently measured by the amount of size/mass gain desired in the larva—that is, by measuring size/mass gain in order to achieve a goal larval biomass production level. By way of example, the larvae may be incubated with the liquid nutrient source for a sufficient length of time to increase in mass by 10%, by 20%, by 30%, by 40%, by 50% or more, including a 2-fold increase in mass, a 3-fold increase in mass, a 4- or 5-fold increase in mass, and so forth.

In some examples, the period of time that the larvae are incubated with the liquid nutrient source is determined based on the growth stage of the larvae. For example, a larva can be incubated with the liquid nutrient source until it reaches the pre-pupa stage, or pupa stage. When at least two larva are incubated with the liquid nutrient source, the larvae can be incubated with the liquid nutrient source until at least one larva reaches a particular growth stage, or all larvae reach the particular growth stage, for example the pre-pupa stage. In some embodiments when larvae are incubated with the liquid nutrient source, the larvae are incubated for a period of time until at least about 10%, 20%, 30%, 40%, 50%, 60%, 70%, 80%, or 90% of the larvae have reached a particular growth stage, for example the pre-pupa or pupa stage. The skilled artisan is familiar with identifying a particular growth stage of a larva.

In some examples, the period of time that the larvae are incubated with the liquid nutrient source is determined based on the size of the larvae. For example, a larva can be incubated with the liquid nutrient source until it reaches a particular size. When at least two larvae are incubated with the liquid nutrient source, the larvae can be incubated with the liquid nutrient source until at least one larva reaches a particular size, or all larvae reach the particular size, for example 0.3 cm in diameter. In some embodiments when at least two larvae are incubated with the liquid nutrient source, the larvae are incubated for a period of time until at least about 10%, 20%, 30%, 40%, 50%, 60%, 70%, 80%, or 90% of the larvae have reached a particular size, for example 0.3 cm in diameter. Larvae approaching the pupae stage reach a size in excess of 2 cm in length and 0.4 cm in diameter relative to immature larvae which start out on hatching from eggs at less than 0.2 cm in length and less than 0.1 cm in diameter. Thus, various sizes in between these ranges, or even more or less than these ranges, may be used to determine the period of time that the larvae are incubated with a liquid nutrient source. For example the larvae may be incubated with a liquid nutrient source until they reach about 0.3, 0.4, 0.5, 0.6, 0.7, 0.8, 0.9, 1.0, 1.5, or 2.0 cm in length, or about 0.2, 0.3, 0.4, or 0.5 cm in diameter. The skilled artisan is familiar with identifying a particular size of a larva.

In some examples, the period of time that the larvae are incubated with the liquid nutrient source is determined based on the organic solute concentration in the liquid nutrient source, for example, the concentration of a particular organic solute, such as protein. For example, a larva can be incubated with the liquid nutrient source until the organic solute concentration reaches a particular level, or until the organic solute concentration is reduced by a particular percentage. A reduction in the organic solute concentration can be measured by determining the organic solute concentration of the liquid nutrient source before incubation with the larvae at various times during incubation with the larvae and after incubation with the larvae. Additionally, the organic solute concentration of the liquid nutrient source may be continuously monitored before, during and after contact with the larvae. The period of time that the larvae are incubated with the larvae can be adjusted in response to the measured organic solute concentration. In some instances, the larvae are incubated with the liquid nutrient source for a period of time until a particular reduction in the concentration of an organic solute concentration is reached, for example an about 1%, 2%, 3%, 4%, 5%, 6%, 7%, 8%, 9%, 10%, 15%, 20%, 30%, 40%, 50%, 60%, 70%, 80%, 90% or even 100% reduction in the concentration of the organic solute. In some embodiments, the larvae are incubated with the liquid nutrient source for period of time until a particular reduction in the concentration of two or more organic solutes is reached. In some embodiments, the larvae are incubated for a period of time until one organic solute concentration is reduced by a particular amount, and another organic solute concentration is reduced by a different amount. The skilled artisan understands how to measure organic solute concentrations.

The skilled artisan will appreciate that certain variables may be considered to optimize the rate of organic solute removal or production of larva biomass, including the chemical properties of the organic solute of interest to be removed from the waste stream, the efficiency of the larvae in assimilating specific organic solutes, the average age and density of larvae in the liquid waste stream, the period of time the larvae are incubated with the organic solute, and the depth, temperature and pH of the liquid nutrient source. The skilled artisan will appreciate that these variables can be adjusted to optimize removal of organic solute from the liquid nutrient source, or production of larva biomass, based upon a comparison of the amount of organic solute, including particular organic solutes present in the liquid nutrient source before and after incubation with the larvae. Hence, the depth of liquid, density of larvae, period of time, etc., can be adjusted as needed in maximizing organic solute removal or production of larva biomass.

### Harvesting Larvae

In some embodiments, larvae are harvested after incubation with the liquid nutrient source. The skilled artisan will understand methods of harvesting larvae, including methods of harvesting larvae from a liquid nutrient source. For example, harvesting larvae includes collecting larvae by hand or by machine, among other methods. Harvested larvae can be dried (if need be for shipping and storage), for example before sale as animal feedstock.

In some examples, the larvae are harvested from the liquid nutrient source following the period of time for incubating the larvae with the liquid nutrient source, as described herein. For example, the larvae may be harvested when the organic solute concentration reaches a particular level, when the organic solute concentration has been reduced by a particular amount, when the larvae reach a particular size, when the larvae reach a particular growth stage or after a designated period of time that the larvae are incubated with the liquid nutrient source. In additional embodiments, the larvae self-harvest from the liquid nutrient source (i.e., the larvae migrate out of the liquid nutrient source) and are then harvested from the position migrated to.

In particular embodiments, harvesting takes place when at least one larva or all larvae reach a particular size, for example 0.3 cm in diameter. In some embodiments when at least two larva are incubated with the liquid nutrient source, the larvae are incubated for a period of time until at least about 10%, 20%, 30%, 40%, 50%, 60%, 70%, 80%, or 90% of the larvae have reached a particular size, for example the 0.3 cm in diameter.

In some embodiments, harvesting the larvae involves passing the liquid nutrient source in which the larvae are incubating through a filter. Larvae approaching the pupae stage reach a size in excess of 2 cm in length and 0.4 cm in diameter relative to immature larvae which start out on hatching from eggs at less than 0.2 cm in length and less than 0.1 cm in diameter. Thus, various filter sizes may be used to harvest larvae, depending on the size of larvae to be harvested. For example, filters with pore sizes of about 0.2, 0.3, 0.4, or 0.5 cm in diameter, or smaller or greater pore sizes may be used to harvest larvae.

Thus, larvae of particular sizes can be periodically harvested from the liquid nutrient source by using a filter with appropriate pore size (e.g., 0.3 to 0.5 cm), leaving smaller larvae (i.e., immature larvae) in the liquid nutrient source. In other embodiments, larvae can be periodically harvested by passing the waste stream through continuous flow centrifugal processing equipment. Filters and continuous flow centrifugal processing equipment are known to the skilled artisan.

Alternatively, the larvae may be constantly harvested. For example, mixed populations of larvae of varying ages, size and/or growth stage may be used in the methods described herein, and larvae can be continuously added and removed from the apparatus as necessary in maintaining operation of the apparatus. In some embodiments, the larvae are constantly harvested by constantly cycling the liquid nutrient source that the larvae are incubating in through a filter or continuous flow centrifugal processing equipment.

### Synergistic Interactions

In some embodiments, use of microbes in combination with larvae, and the inclusion of nutrients beneficial to the well-being of the larvae, may be expected to enhance larval capacity to process a liquid nutrient source stream. Thus the methods disclosed herein, and the apparatus for use with these methods, and its optimization in processing a liquid nutrient source stream, include exploration of optimal conditions that depend upon the nature of the material being processed. This includes pH, chemical composition, synergistic interactions that may be exploited such as the presence or absence of microbes in the liquid nutrient source stream, the period of time the larvae are incubated with the liquid nutrient source, etc. As noted earlier, optimization can be measured by comparing the organic solute concentration, including concentration of particular organic solutes before and after incubation with the larvae by any of a variety of measuring techniques known to the skilled artisan.

Synergism between larval processing of liquid wastes and microbes present or growing in the liquid waste may occur, e.g., when a specific microbe, itself growing in the liquid waste, provides a nutrient source that serves as a feed source for the larvae. Alternatively, synergism between larvae and microbes present in the liquid waste may arise as a result of microbes producing vitamins, essential amino acids, or lipids (or other compounds) as byproducts of the microbes' growth in the liquid, one or more of which compounds stimulate the growth of the larvae. Synergism in some embodiments results in the removal of byproducts present in the liquid waste which the microbes alone cannot clear from the liquid.

### VI. Representative Apparatuses for Performance of Methods Described Herein

Provided herein are apparatuses and devices that can be used to perform the disclosed methods of removing organic solute from a liquid nutrient source and increasing larva biomass.

The apparatus disclosed herein includes an enclosed tank, comprising a liquid entry port, a liquid exit port, a gas entry port, a gas exit port, a gas pump, as well as, an inner reservoir capable of holding liquid, wherein the liquid entry and exit ports are operably linked to the inner reservoir, and access means for accessing the inner reservoir of the tank. The enclosed container is made of a substance that is substantially impermeable to air, for example, metal, rubber, wood or plastic. The enclosed tank may be any shape or size, for example the enclosed tank may have a box configuration. The skilled artisan will understand methods and materials for the construction of the enclosed tank.

The apparatus includes liquid entry and exit ports and gas entry and exit ports. Such ports will be familiar to the skilled artisan. The ports described herein form a gas impermeable seal with the enclosed tank. Such seals are also familiar to the skilled artisan.

In some embodiments, the apparatus includes means for maintaining the interior of the enclosed tank at a negative air pressure relative to the external air pressure. For example, in some embodiments an air pump is positioned inside enclosed tank, drawing air from inside the tank and forcing it outside the tank through the gas exit port. Alternatively, the air pump can be positioned outside the enclosed tank with its air intake communicating inside the enclosed tank so as to maintain a net negative air pressure inside the enclosed tank relative to the external air pressure. In such embodiments, maintenance of negative air pressure inside the enclosed tank is important so that volatile greenhouse gases and odors can be drawn through the air pump and directed subsequently to scrubbing tanks designed to remove these components from the gas stream before the air, cleared of these elements, is vented to the outside atmosphere.

The apparatus includes an inner reservoir capable of holding a liquid nutrient source. For example, the inner reservoir may have an open box configuration. In some embodiments, the inner reservoir has a box configuration, including a lid. The liquid entry and exit ports are operably linked to the inner reservoir such that liquid nutrient source can flow from the liquid entry port, into the inner reservoir, and out the liquid exit port. In some embodiments, the inner reservoir forms a serpentine path for the flow of liquid nutrient source between the liquid entry and exit ports. One of skill in the art will be familiar with methods and materials for the construction of the inner reservoir.

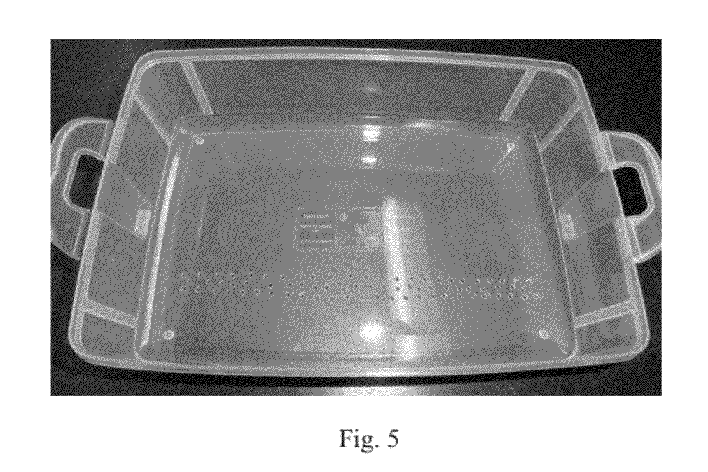

In some embodiments, the inner reservoir comprises one or more arrays of interconnected modular larvae incubation units (MLIUs). Any number of MLIUs may be included in each array. The MLIUs are made of a substance that is substantially impermeable to liquid, for example, metal, rubber, wood or plastic. In some embodiments, the uppermost module in an array of MLIUs comprises a lid. MLIUs are further described herein (e.g., see FIG. 5). Additionally, the skilled artisan will understand methods and materials for the design and construction of MLIUs.

In some embodiments, the MLIUs are included in an array of MLIUs, wherein the MLIUs are positioned such that at least one MLIU is above at least one other MLIU. Any number of MLIUs may be included in an array of MLIUs. In some cases, multiple MLIUs will be located above one or more MLIUs. An array of MLIUs may include MLIUs stacked directly above one another, or may include MLIUs stacked partially above one another, or a combination thereof. Some MLIUs are constructed to be capable of connecting to other MLIUs, others are not capable of connecting to other MLIUs. For example, an array of MLIUs may include MLIUs capable of interconnecting vertically to form vertical arrays of MLIUs, e.g., by snapping together snuggly one on top of the other in vertical stacks of repeating units.

In each array of MLIUs, liquid nutrient source may pass from higher MLIUs to lower MLIUs. Each MLIU in an array of MLIUs (except the lowest unit or units in an array) includes means for liquid to pass to a lower MLIU in the array, e.g., an MLIU may include drain holes spanning longitudinally across the floor of the module which allows a liquid nutrient source passing through the module to drain into an identically constructed module positioned under the upper module to catch the liquid nutrient source passing into it from the overhead module. In some embodiments, the drain holes are up to about 0.2 cm in diameter, or even larger. The drain holes may be positioned anywhere along the floor of a modular unit. In some embodiments, the drain holes are positioned to allow a temporary shallow reservoir of liquid nutrient source to accumulate in the area free of drain holes, e.g., by positioning the drain holes in the base floor of the MLIU approximately one-quarter of its floor width from wall to wall. The lowest MLIU in an array of MLIUs usually lacks drain holes, allowing liquid nutrient source traveling through the array of MLIUs to collect at least temporarily in the lowest unit.

The angle of an array of MLIUs may be tilted from a flat horizontal position before or while a liquid nutrient source passes through the array, thereby allowing for an increase or decrease (depending on the angle) in the depth of liquid nutrient source in the area free of drain holes.

In some embodiments involving MLIUs, the modular units include holes in the side walls of the units. For example, holes may be included in the sidewalls up to about 0.2 cm in diameter, or even larger. The holes provide for air in supporting respiration of any BSF larvae housed in the modular units. For example, the holes may be placed about 0.5 to 2.5 cm beneath its top rim of a modular unit.

In embodiments including an inner reservoir comprising one or more arrays of MLIUs, the liquid entry port is operably linked to at least one MLIU that is higher than the lowest MLIU in the one or more arrays of MLIUs. The liquid exit port is operably linked to at least one of the MLIUs. For example, the liquid entry port may be operably linked to the highest MLIU in the one or more arrays of MLIUs and the liquid exit port may be operably linked to the lowest MLIU in the one or more arrays of MLIUs. In embodiments including more than one array of MLIUs, one or more MLIUs in an array of MLIUs may be operably linked to one or more MLIUs in the same or a different array of MLIUs, for example by connecting the MLIUS with a tube.

In some embodiments, the lowest module in an array of MLIUs is operably linked to a tube which is connected to a MLIU that is higher than the lowest the uppermost module(s) in the array, thereby allowing recycling of the liquid nutrient source from the lowest module to the uppermost module using a circulating pump designed to feed the liquid back into the upper unit of the array.

In embodiments including an inner reservoir comprising multiple arrays of MLIUs, the uppermost module(s) in the uppermost array of modular units is operably linked to the liquid entry port. The lowest module in the uppermost array of modules lacks drain holes and is operably linked to the uppermost module in the second-highest array. The lowest module in the second highest array is operably linked to the uppermost module in the next highest array, etc. The lowest module in the lowermost array of MLIUs is operably linked to the liquid exit port. In some embodiments, the lowest module in an array is operably linked to a tube which is connected to the uppermost module in the array, thereby allowing recycling of the liquid nutrient source from the lowest module to the uppermost module using a circulating pump designed to feed the liquid back into the upper unit of the modular stack.

For example, to start the processing and cascading of liquid nutrient source through the units, larvae are first added to each MLIU (except the lowermost MLIU), and liquid nutrient source is then allowed to infuse into the top MLIU via the liquid entry port. The liquid begins to fill the top MLIU, and as it reaches the drain holes, trickles into the MLIU snapped into position below it, in turn filling up to the drain holes of the second MLIU, passing then to the unit below, etc. Larvae can be added to and harvested relatively easily from the individual MLIUs by simply opening and closing the snap-lock connections. Furthermore, the drain holes in the modules make it possible to use the MLIUs as sieves in washing the larvae free of liquid nutrient source at harvest.

The apparatus includes access means for accessing the inner reservoir of the enclosed tank. In some embodiments, the access means has opened and closed states. When open, the access means allows for access to the inner reservoir of the apparatus. When closed, the access means forms a seal with the enclosed tank and does not allow access to the inner reservoir of the apparatus. The seal is substantially impermeable to air. For example, in some embodiments, the access means may be a lid or door that opens and closes, allowing access to the inner reservoir of the apparatus when in the open state.

In some embodiments, the apparatus includes means for maintaining larvae at a depth of about 0.5 to about 3 cm from the top surface of the liquid nutrient source. In some embodiments, the depth of liquid nutrient source accumulating in the inner reservoir may be determined by how high above the floor of the inner reservoir the operable linkage to the liquid exit port is positioned. For example, the height may be set at 0.5 to 3 cm above the floor of the inner reservoir, thereby maintaining the larvae in the inner reservoir at a depth of about 0.5 to about 3 cm of liquid nutrient source. Some embodiments include an operable linkage to the liquid exit port or a liquid exit port that is adjustable. These embodiments allowing for adjustment of the height of the operable linkage to the liquid exit port or liquid exit port over the floor of the inner reservoir; thus allowing for adjustment of the depth of liquid nutrient source in the inner reservoir. In other embodiments, a pump is used to maintain the level of liquid nutrient source in the inner reservoir. Alternatively, means for adjusting the flow of liquid nutrient source into or out of the inner reservoir may be included to maintain the height of the liquid in the inner reservoir, for example by including a valve at the liquid entry or exit ports. In some embodiments, the depth of liquid nutrient source in the inner reservoir can be set at more than 3 cm, providing for a larger processing reservoir. In this instance, a net or screen having a pore size small enough to contain immature larvae (i.e., less than 0.1 cm in diameter) is included in the apparatus at about 0.5 to about 3 cm from the top of the liquid nutrient source. The height of the net or screen may be adjustable, for instance to maintain a constant larva depth when the depth of the liquid nutrient source is altered.

In some embodiments, a means for preventing larvae from passing through the liquid exit port is included. For example, a filter positioned at the inner reservoir or at the liquid exit port or in between the inner reservoir and the liquid exit port keeps larvae from escaping the apparatus as liquid exits the apparatus. Such a filer has a pore size of less than 0.1 cm in diameter (i.e., a pore size smaller than an immature larva).

In some embodiments, the apparatus includes means for monitoring the organic solute concentration of the liquid in the liquid entry port, in the inner reservoir, in the liquid exit port, or a combination of two of more thereof. For example, the apparatus may be designed for samples of liquid to be removed for testing and measurement of organic solute concentration as described herein. In other examples, the apparatus includes instruments capable of detecting organic solute concentration. Such instruments are known to the skilled artisan.

In some embodiments, the apparatus includes means for recycling a liquid nutrient source through the apparatus. For example a pump and piping system may be included to cycle liquid exiting the liquid exit port back the liquid entry port.

In some embodiments, the apparatus includes a means for removing larvae from the inner reservoir (e.g., means for removing mature larvae and those entering the pupa stage). For example, in some embodiments, the means for accessing the inner reservoir is sized appropriately to allow mechanical removal of larvae (e.g., scooping of the larvae by hand). In other embodiments, the apparatus includes a vacuum capable of removing larvae from the inner reservoir. Other embodiments include a filter or screen with a pore size that allow immature larvae to pass, but not mature larvae (e.g. a pore size of 0.3 to 0.5 cm in diameter). In some such embodiments, the filter is positioned in the inner reservoir such that it can be passed through the liquid nutrient source in the inner reservoir.

In some embodiments, the apparatus is operably linked to an air pollution control device, such as a gas scrubber. For example, a gas scrubber may be operably linked to the gas exit port. The use and manufacture of gas scrubbers is known to those of skill in the art; gas scrubbers are available commercially. For example, wet, dry and semi-dry gas scrubbers may be used, among others. Thus, a train of Ca(OH)₂ and acidic (H₂SO₄) sparging towers, biofilters, bio-nitrification towers, wet electroscrubbers or other devices may be used as a gas scrubber for purging and scrubbing gases (such as VOAs, volatile amines, CO₂, N₂O, etc.; see, e.g., Schifftner and Hesketh, Wet Scrubbers (2^{nd} Ed.), Lancaster: Technomic Publishing, 1996; Devinny et al., Biofiltration for Air Pollution Control, Boca Raton: Lewis Publishers, 1999; Jaworek et al., Environ. Sci. Technol., 40:6197-207, 2006; Amlinger et al., Waste Manag. Res., 26:47-60, 2006; U.S. Pat. No. 6,013,512).

In some embodiments the apparatus includes means for cooling or heating the liquid nutrient source in the inner reservoir. For example, the apparatus may include a heating element that heats the liquid nutrient source before it enter the inner reservoir, for example the heating element may be positioned at the liquid entry port. In other examples, the apparatus may include a heater that warms the interior of the apparatus. Additionally, the apparatus may include an air conditioning unit to cool the interior of the apparatus.

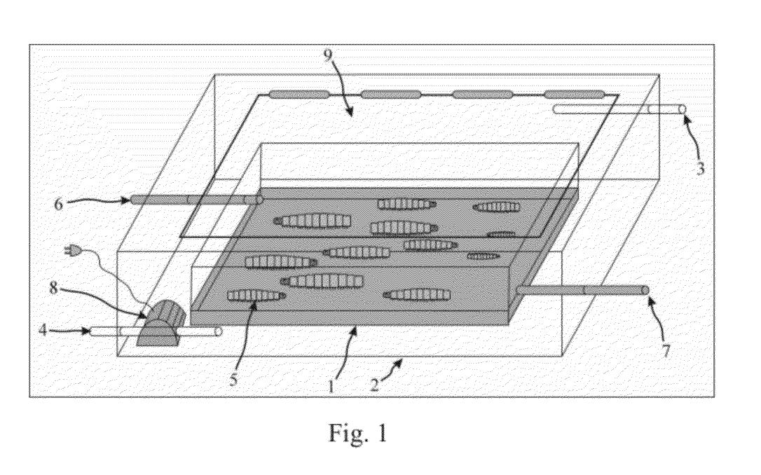

FIG. 1 illustrates an embodiment of the apparatus having an open inner reservoir 1 for processing a liquid nutrient source enclosed inside enclosed tank 2. Enclosed tank 2 is made of material substantially impermeable to gases, has a gas entry port 3 where air is drawn inside enclosed tank 2, and a gas exit port 4, where air exits enclosed tank 2. Airflow through enclosed tank 2 provides larvae 5 with air needed for respiration as they incubate with the liquid nutrient source passing through inner reservoir 1. A liquid nutrient source stream is introduced into inner reservoir 1 by way of a liquid entry port 6 passing through the wall of enclosed tank 2, filling inner reservoir 1 to a depth of about 0.5 cm to about 3 cm liquid nutrient source. A liquid exit port 7 provides a means for larvae-processed liquid nutrient source to exit the apparatus. The shallow liquid depth in inner reservoir 1 allows larvae to respire as they feed on the liquid nutrient source. Enclosed tank 2 is constructed with gas entry port 3 near its topside so that entering air mixes with gases given off during fermentation and microbial and BSF respiration inside enclosed tank 2. Air is forcibly drawn inside enclosed tank 2 through gas entry port 3 by slightly lowering the air pressure in enclosed tank 2 relative to the external air pressure. In some embodiments, as illustrated in FIG. 1, an air pump 8 draws air from inside enclosed tank 2 and forces it out exit port 4, is included. Alternatively, the air pump can be placed outside the enclosed tank 2. However, in the latter instance, the air intake for the air pump must be communicated inside the enclosed tank so that the net air pressure inside the enclosed tank remains negative relative to the external air pressure.

### EXAMPLES

The following examples are provided to illustrate certain particular features and/or embodiments. These examples should not be construed to limit the invention to the particular features or embodiments described.

### Example 1

### BSF Larvae Clear Alcohols and VOAs from Compost Tea

This example illustrates that BSF larvae clear short chain alcohols and VOAs from compost tea. Concentrations of ethanol, propanol, acetic acid, propanoic acid, butyric acid and isovaleric acid were measured in control and BSF-larvae treated compost tea. The results show that BSF larvae have the capacity to specifically clear short chain alcohols and VOAs from compost tea.

Compost tea derived following fermentation of food scraps consisting of left over coffee grounds, produce, discarded bread, cooked rice, fish bones and skin, egg shells, etc. formed by enclosure of the food scrap in a 20 L incubator maintained at ambient room temperature (˜21° C.) for one month was drawn into cylindrical polyethylene reservoirs set up in triplicate and tilted at 20° angles from a horizontal plane to create a shallow layer of liquid nutrient source in the reservoir open to room air at the surface layer, approximately 1 cm deep at the deepest section of the reservoir and providing a means for BSF larvae (2 per ml liquid nutrient source) to migrate in and out of the liquid nutrient source as they feed. An identical set of reservoirs were set up with compost tea drawn from the same stock but lacking BSF larvae, and all of the reservoirs were placed in an incubator maintained at 30° C.

Aliquots (10 μl) of the liquid nutrient source were drawn and analyzed in duplicate for short chain alcohol and VOA concentration using a 30 m×0.24 mm ID Stabilwax-DA capillary column on a Shimadzu GC-2010 gas chromatograph equipped with a splitter (50:1 setting), flame ionization detector and using the following temperature profile setup: injector at 240° C., iso 60° C. for 10 min., ramp 10° C. per min. to 240° C. and detector at 260° C. Results were expressed in mM VOA by measuring peak areas and comparing peak areas for each alcohol and VOA analyzed to that of authentic standards made up in known concentrations. Samples were drawn from each reservoir at the beginning of the experiment, and two days later, and test outcomes compared.

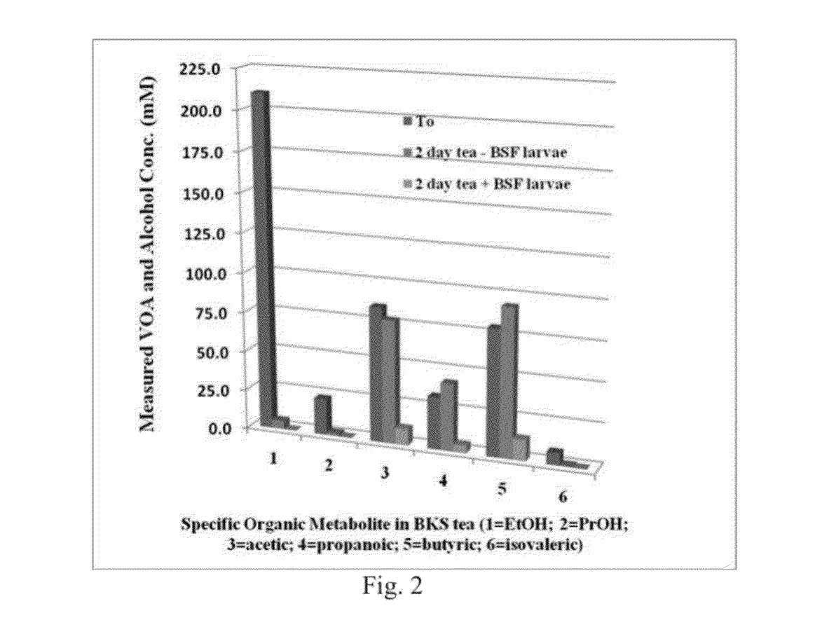

Results are shown in FIG. 2. Addition of BSF larvae to the compost tea markedly accelerated turnover of VOAs in the compost tea evidenced by the sharp fall off in the concentration of the short chain alcohols and all of the principle VOAs detected in the compost tea relative to the control experiments in which BSF larvae were omitted. Concentrations of ethanol (column set 1), propanol (column set 2), acetic acid (column set 3), propanoic acid (column set 4), butyric acid (column set 5) and isovaleric acid (column set 6) were measured. In these studies the baseline concentration of short chain alcohols and VOAs in the compost tea at the beginning of the experiment were as follows: ethanol, 221 mM; n-propanol, 23 mM; acetic acid, 86 mM; propanoic acid, 33 mM, butyric acid, 80 mM, and isovaleric acid 7.7 mM. Valeric and caproic acid, sometimes detected in trace amounts in compost tea, were not present in the compost tea used in these studies. Subsequent results two days later showed that tea containing BSF larvae was completely clear of ethanol, n-propanol, and residual albeit still detectable concentrations of acetic acid (10.5 mM), propanoic acid (4.9 mM), and butyric acid (13.3 mM). The average reduction in residual concentration of alcohols and VOAs in the compost tea having BSF larvae feeding on the tea relative to control tea lacking BSF larvae averaged from 7- to 10-fold. These results establish that BSF feeding on compost tea have the capacity to clear short chain alcohols and VOAs from the liquid nutrient source at a markedly accelerated rate over that which occurs in their absence.

### Example 2

### BSF Larvae Clear Amines and Residual Protein from Compost Tea

This example illustrates that BSF-larvae clear ninhydrin-positive amines from compost tea. Glycine, glycyl-glycine, glutamic acid and leucine were measured in control and BSF larvae treated compost tea by thin layer chromatography. The results of this experiment show that BSF larvae can clear nitrogen metabolites from compost tea.

To verify turnover of amines and residual proteins in compost tea, a similar set of experiments were set up as in Example 1, but instead of measuring alcohols and VOA content, total protein concentration and ninhydrin-positive amines were studied by comparing compost tea treated with BSF larvae to control tea lacking BSF larvae.

Aliquots of BSF larvae-treated tea and untreated control tea were first compared for protein content following a two day larvae exposure period at 30° C. using the standard Bradford protein assay and bovine serum albumin as a protein calibrator. As determined use the Bradford protein assay, protein in control compost tea was 275±7 μg ml⁻¹ (±2 SD; n=3) compared to 148±13 μg ml⁻¹ (±2 SD; n=3) in compost tea processed with BSF larvae, indicating that the BSF larvae while feeding on the compost at 30° C. cleared ˜50% of the protein from the compost tea relative to control tea in which BSF larvae were excluded.

Aliquots of compost tea derived from control and BSF larvae-treated samples were also examined for changes in ninhydrin-positive amines using thin layer chromatography silica G plates (20×20 cm; 250 μM thick). Ninhydrin standards of known amino acids (glycine, glycyl-glycine, glutamic acid and leucine) were applied to the plate in 20 μl aliquots and run simultaneously with aliquots of control and BSF-larvae treated compost tea drawn from 14K, 5 minute supernatant fractions recovered from a microfuge also applied to the same plates. Amines were separated using a solvent made up to 90% isopropanol and 10% H₂O, and after air drying thin layer plate the plate was sprayed with ninhydrin reagent (2% made up in acetone), and heated in a 100° C. oven for ˜30 minutes to detect amines present in the samples applied to the plate

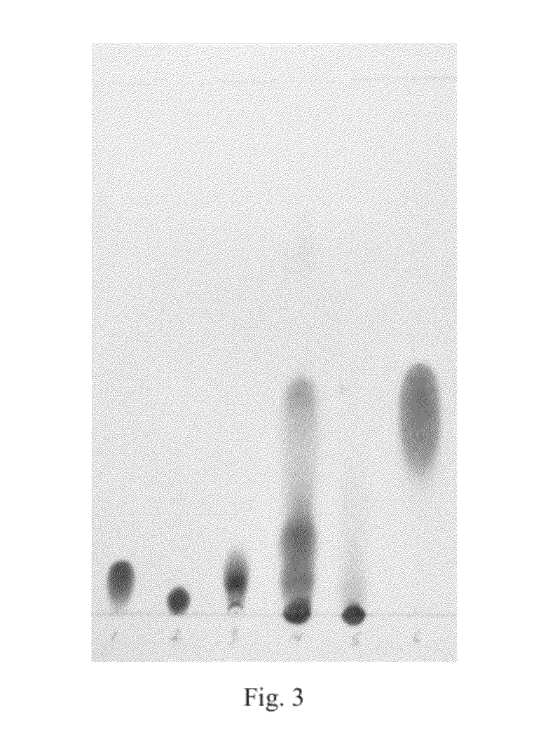

The results of the amine assays are shown in FIG. 3, which shows a comparison of the ninhydrin-positive pattern recovered from the thin layer chromatography plate for standards, control and BSF larvae-treated compost. Samples were applied to the plate as follows (left to right): lane 1, glycine; lane 2, glycyl-glycine; lane 3, glutamic acid; lane 4, compost tea control; lane 5, compost tea processed with BSF larvae; lane 6, leucine. The larvae-treated tea was substantially cleared of amines except for positive material remaining at the point of application. The latter is likely residual protein remaining in the tea which stains positive with ninhydrin reagent but is too polar to migrate from its point of application on Silica gel G plates.

Together, the results of the protein and amine assays confirm that BSF larvae incubated with compost tea markedly reduced nitrogen metabolites in the tea as reflected by the decrease in total protein content and ninhydrin-positive metabolites relative to control compost tea treated similarly but without BSF larvae feeding on the tea.

### Example 3

### BSF Larvae Grow and Assimilate Carbon, Nitrogen and Other Essential Elements and Nutrients while Feeding on Various Liquid Nutrient Sources

This example illustrates that BSF larvae grow and assimilate carbon, nitrogen and other essential elements and nutrients while feeding on a variety of filtered liquid nutrient sources. The larvae were allowed to feed freely on: urine, sewage water (7 fold diluted), water extract of Gainesville House Fly diet (Sheppard et al., J. Medical Entomology, 39:695-698, 2002), chicken broth, orange juice, compost tea (prepared as in Example 1), water extract of chicken manure ((200 g DW chicken manure extracted in 2 L tap water at 20° C.), commercial 2% milk and whey from soured 2% milk. The weight gain of the larvae was measured over the course of 7 days. The results of this experiment establish that BSF larvae feeding solely off liquids with organic solutes thrive and gain weight. In compost tea the larvae were grown in separate experiments to full maturity.

Since BSF larvae attain a protein content of ˜40+%, and total lipid content in the range of ˜30+% in growing on solid nutrient sources as they mature into the pupate stage, experiments were set up to measure their ability to grow and assimilate carbon and nitrogen required for protein synthesis and lipid production by tracking their growth on compost tea compared to that of control larvae fed solid food scraps.

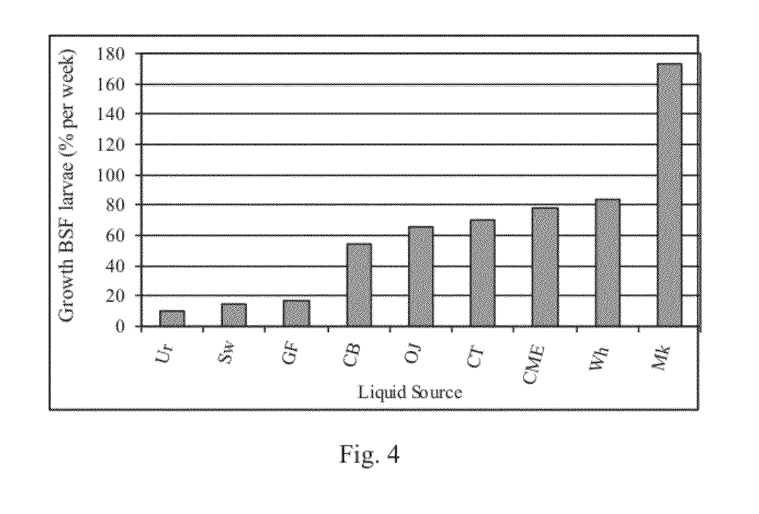

The results of these experiments are shown in FIG. 4. BSF larvae grew on all liquids tested. Larvae feeding on compost tea continued to gain weight throughout the full 30 days of the experiment and by the 30^{th} day reached a comparable weight to that of larvae fed food scrap. At this weight, the BSF larvae fed compost tea were entering the pupate stage, and the experimental measurements were terminated.

These results demonstrate the ability of BSF larvae to assimilate secondary metabolites present in liquid nutrient sources since their biomass consists of proteins, carbohydrates, DNA, lipids, etc., compounds that could only have been drawn from essential elements and nutrients made up of carbon, nitrogen, sulfur, and phosphorous required as a prerequisite for their growth and survival over the duration of these test as they fed on the liquid waste.

### Example 4

### BSF Larvae Process VOA Solutes Dissolved in Particulate-Free Liquid

This example illustrates that BSF larvae can process dissolved VOA in a solution of only a single VOA and water. Tubes containing solutions of ˜0.9% v/v butyric, acetic or valeric acid were incubated at 30° C. for approximately one week with and without BSF larvae. Acid concentrations were measured by gas chromatography and used to calculate the turnover rate for each of the VOAs. The results show that each of the VOAs was processed by the BSF larvae, demonstrating that the ability of BSF larvae to process solutes in an aqueous solution and that BSF larvae have the capacity to turnover organic acids commonly found in abundance in decomposing organic matter independent of solids suspended in the liquid nutrient source stream.

Solutions of ˜0.9% v/v butyric, acetic or valeric acid and chlorinated tap water were dispensed in paired sets in Hungate culture tubes (capped with a polyurethane open pore plug) similarly as in Example 1. Larvae were omitted from one set of tubes labeled controls, and BSF larvae (approximately 2 weeks old) were added to the other matching set. The tubes were incubated at 30° C. for approximately one week and the concentration of each organic acid was tracked by gas chromatography. During this interval the larvae remained active evidenced by swimming and spiraling about in the liquid and crawling up and down the walls of the Hungate tubes.

From the concentration measurements, the turnover of each VOA was determined (evidenced by a fall in organic acid content as BSF larvae fed on the solution relative to the corresponding organic acid content of the controls). The turnover of butyric, acetic and valeric acids was calculated from the difference in VOA concentrations between the two parallel sets of tubes for each solute tested (control residual organic acid content less that of the corresponding BSF larvae test set) with results expressed as VOA turned over in mmoles L⁻¹ day⁻¹ per 10³ larvae. The larvae weight was approximately 100-150 mg each. Table 1 shows the results of these calculations. These results show that BSF larvae have the capacity to turnover organic acids commonly found in abundance in decomposing organic matter independent of solids suspended in the waste stream.

TABLE 1
BSF larvae processing of organic acid from particulate free water
containing only one organic acid.

| | Turnover Activity |
|---|---|
| VOA | (mmoles L⁻¹ day⁻¹ per 10³ larvae) |
| Butyric acid | 27 |
| Acetic acid | 72 |
| Valeric acid | 19 |

### Example 5

### Operation of a Representative Apparatus for Removing Organic Solute from a Liquid or for Producing Larva Biomass

This example illustrates construction and operation of an apparatus comprising an array of modular larval incubation units (MLIUs) as provided for herein.

The modular units are constructed from identical polyethylene rectangular boxes designed to snap together snuggly one on top of the other in a vertical stack of repeating units in which larvae are retained for incubation with compost tea. Each modular unit, with the exception of the lowest module in the stack of modules has drain holes (0.2 cm in diameter) spanning longitudinally across floor of the module which allows compost tea passing through the module to drain into an identically constructed module positioned under the upper module to catch the fluid passing into it from the overhead module. FIG. 5 is an example of how the drain holes in an upper module are placed into the floor of the module. By positioning the drain holes in the base floor of the modular unit some distance from the edge of the unit's side walls, in this case approximately one-quarter of its floor width from wall to wall, a temporary shallow reservoir of compost tea accumulates in the area free of drain holes, on which BSF larvae can feed.

BSF larvae were added to each unit, and compost tea allowed to flow into the top unit by means of a pump and tubing carrying the compost tea to the top module. As the compost tea began to accumulate in the top module, it reached the drain holes, trickled into the module snapped into position below the top module, in turn filling up to the drain holes of the second unit, passing then to the unit below, etc., until finally collecting in the bottom unit. The bottom unit lacks drain holes similar to the other units, but contains a tube and drain assembly providing a means of removing processed compost tea from the modular stack out of the invention for subsequent handling. At this point liquid can be either recycled through the stacked units using a circulating pump designed to feed the liquid back into the upper unit of the modular stack, or carried away from the units in a single pass through operation.

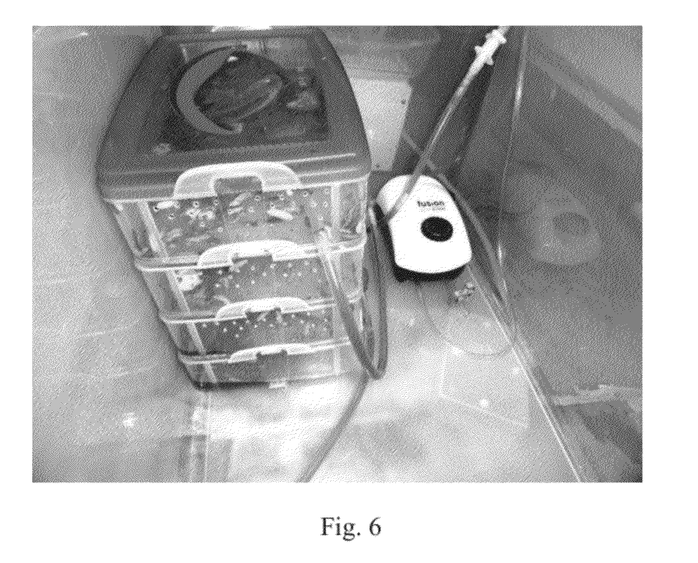

As shown in FIG. 6, to provide for air in supporting respiration of the BSF larvae housed in the modular units, multiple holes, each about 0.2 cm in diameter, were placed around the upper perimeter walls of each modular unit on three of the unit's walls from about 0.5 to 2.5 cm beneath its top rim.

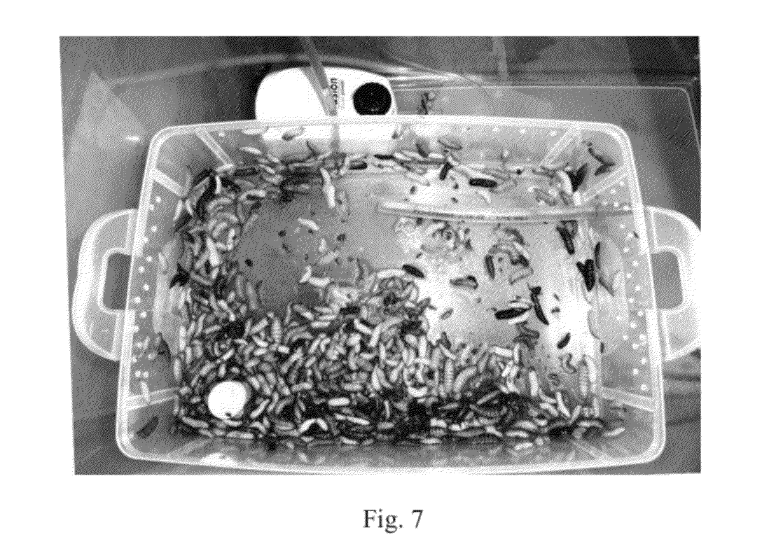

FIG. 6 shows a side view of a series of operating modular units housed inside an enclosed tank as illustrated in FIG. 1. Larvae are present inside each unit (except for the bottom liquid collection unit), feeding on compost tea. Breathing holes can be seen in each of the modular processing units (excluding the bottom liquid collection unit). The modular units, snapped together as in FIG. 6, rest on the floor of the outer box reservoir. The outer box reservoir is furthermore tilted at an angle of approximately 20°, causing compost tea infusing into each modular reservoir to temporarily pool in its lower left basin area as it trickles and flows through the units, allowing BSF larvae retained in the modules to feed on the compost tea as it works its way to the collection module at the bottom of the stack. FIG. 7 shows a top down view of the top module making up a modular stack with its lid removed housing BSF larvae and compost tea delivered into the unit from the infusion inlet port illustrated in FIG. 1.

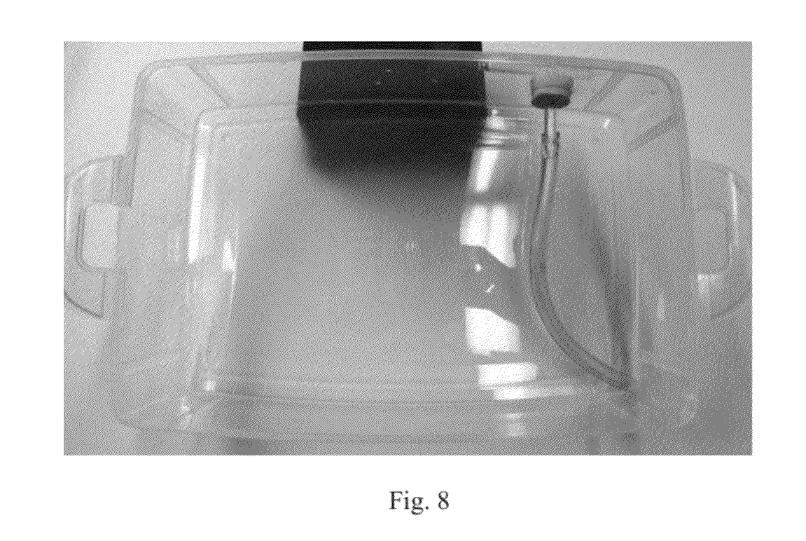

FIG. 8 shows the bottom module used in the apparatus shown in this example. The bottom modules is equipped with a drainage tube allowing fluid passing into its reservoir to be drawing out through the liquid exit port as illustrated in FIG. 1. The purpose of the bottom module in the stacked modular is to provide a means of collecting processed liquid nutrient source and a mechanism for then passing it out the exit port illustrated in FIG. 1.

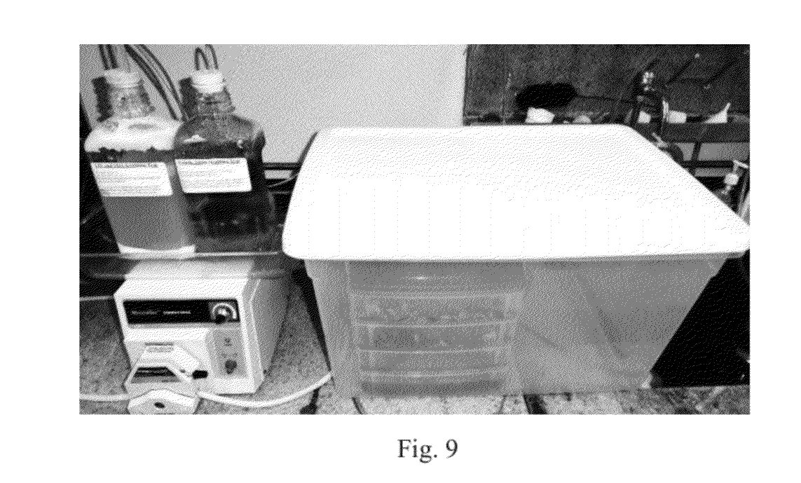

FIG. 9 shows the assembled and operating apparatus as described in this example, including the stacked modules housed inside the enclosed tank with compost tea entering the device by means of a liquid peristaltic pump passing tea into the enclosed tank a liquid entry port and into the uppermost module of the inner reservoir. BSF larvae were present in each module of the array and were incubated with the compost tea passing through the apparatus. The BSF larvae were added through the lid of each module residing above the collection module at the bottom of the stack. Processed compost tea collected at the bottom of the modular stack passes out of the bottom module and exits the enclosed tank through the liquid exit port inserted in the wall of the enclosed tank.

A vacuum air pump operating inside the enclosed tank and operably connected to a gas exit port maintains a negative air pressure inside the enclosed tank and directs the flow of gases drawn into the enclosed tank, and those generated by metabolic activity through a gas exit port. Gases exiting the enclosed tank through the gas exit port pass directly through sparging tank reservoirs designed to capture residual CO₂, VOAs and volatile amines.

### Example 6

### Temperature and pH Tolerance of BSF Larvae Feeding on Compost Tea

BSF larvae feeding on compost tea larvae tolerated compost tea well at ambient room temperature conditions (approximately 21° C.) and up to 35° C. Between 35° C. and 40° C., BSF larvae lost considerable vigor and became extremely sluggish, lost weight, showed excessive molting of their outer exoskeleton, stiffened up and died. In cold tap water (approximately 10 to 15° C.), the larvae stiffen up and tend to become immobile, but on warming back up to ambient room temperature conditions resume their normal writhing and crawling activities as they feed on compost tea.

BSF larvae feeding well in processing liquid nutrient sources exhibit a characteristic almost nonstop continuous writhing, rolling, and crawling activity. Evidence that the larvae are thriving on the liquid waste can be easily viewed by their continuous movement and crawling activities as they feed off the liquid concomitant with weight gain and a transition in the color of their pigmented exoskeleton from an initial white-tan yellow color after hatching from eggs to a darker reddish-brown to black-brown color as they mature into their 5^{th} instar pupa state.

BSF larval behavior as a function of the compost tea pH is similar to the effect of temperature on BSF larvae. The BSF larvae remain very active in writhing, crawling and gaining weight while feeding on compost tea ranging in pH from as low as pH 3.0 to as high as approximately 9.0. No long term decrease in larvae survival or growth rates in compost teas residing in this pH range were observed. However, as the pH of the tea reached 9.4, after a one week interval at this pH the BSF larvae were observed to stiffen up, lose weight, and die off. Concomitant with their dying off, fungi were observed growing on the surface of the compost tea suggesting that at this latter extreme pH range the larvae are not able to efficiently compete for and feed on nutrients in the compost tea with fungi which began to outgrow and outcompete the larvae for the food source. It is possible that the fungi also produce larval toxins which ultimately impair the survival of the larvae this more alkaline pH ranges.

### Example 7

### Effect of Larval Density and Liquid Depth on the Viability of BSF Larvae Feeding on Liquid Compost Tea

BSF larvae added to compost tea incubated at room temperature, and under experiments run at 30° C., did well in terms of their behavioral characteristics while feeding on the tea evidenced by their crawling and writhing activities, and by their growth activity when kept under these environmental conditions. They grew to maturity, reaching the pupa stage, between three and four weeks at the vary latest following introduction to compost tea when maintained with fresh compost tea at least every third day during their growth cycle at a density of 2×10³ L⁻¹. Experiments with larvae densities up to 2×10⁴ L⁻¹ in compost tea were also run successfully evidenced by healthy activity in crawling and feeding of the larvae on compost tea, and clearance of VOAs at shortened intervals as the density of the larvae population increased. The larvae were observed to actually cluster together in close proximity to one another while feeding on the tea as the density increased, and by their behavior to seek out and crawl up and over other larvae in proximity to them as they fed on the liquid. This behavior suggests that the larvae density in liquid waste can be raised to slightly in excess of 2×10⁴ L⁻¹, depending upon the age and size of the larvae. The major limitation appears to be maintenance of sufficient liquid nutrients that the larvae process so as to not starve them and cause die off and putrefaction of the dead larvae.

It will be understood by those of skill in the art that numerous and various modifications can be made without departing from the spirit of the present disclosure. Therefore, it should be clearly understood that the embodiments disclosed herein are illustrative only. We therefore claim all that comes within the scope and spirit of these claims.

We claim:

1. A method of removing organic solute from a liquid, comprising:
selecting a liquid nutrient source containing organic solute, wherein the nutrient source comprises at least 80% water or other liquid and is substantially free of solid nutrients; and
incubating fly larvae for a period of time with the nutrient source, thereby removing organic solute from the liquid.

2. The method of claim 1, wherein the liquid nutrient source comprises compost tea, liquid produced from urine, whey, sewage, liquid produced from manure, or a combination of two or more thereof.

3. The method of claim 1, wherein the pH of the liquid nutrient source is about 3.2 to about 9.4.

4. The method of claim 1, wherein the fly larvae are selected from the group consisting of Musca domestica larvae, Muscina stabulans larvae, Fannia canicularis larvae, Fannia femoralis larvae, Ophyra aenescens larvae, Hermetia illucens larvae or a combination of two or more thereof.

5. The method of claim 4, wherein the fly larvae are Hermetia illucens larvae.

6. The method of claim 1, wherein the fly larvae are incubated:
at about 2×10³ to about 2×10⁵ larvae per liter of liquid nutrient source;
at a temperature of about 20° C. to about 40° C.;
with the liquid nutrient source for about 1, 6, 12, 24 or 48 hours, or about 1 day, 1 week or 1 month; or
a combination of two or more thereof.

7. The method of claim 1, further comprising harvesting the larvae.

8. The method of claim 7, wherein harvesting the larvae comprises:
harvesting mature larvae, or larvae entering the pupa stage of the larva life cycle, or both;
passing the larvae through a filter having a pore size of about 0.3 cm to about 0.5 cm; or
both.

9. A method of producing larva biomass, comprising:
incubating fly larvae for a period of time with a liquid nutrient source containing organic solute, wherein the nutrient source comprises at least 80% water or other liquid and is substantially free of solid nutrients; and
harvesting the fly larvae, thereby producing larva biomass.

10. The method of claim 9, wherein the liquid nutrient source comprises compost tea, liquid produced from urine, whey, sewage, liquid produced from manure, or a combination of two or more thereof.

11. The method of claim 9, wherein the pH of the liquid nutrient source is about 3.2 to about 9.4.

12. The method of claim 9, wherein the fly larvae are selected from the group consisting of Musca domestica larvae, Muscina stabulans larvae, Fannia canicularis larvae, Fannia femoralis larvae, Ophyra aenescens larvae, Hermetia illucens larvae or a combination of two or more thereof.

13. The method of claim 12, wherein the fly larvae are Hermetia illucens larvae.

14. The method of claim 9, wherein the fly larvae are incubated at about 2×10³ to about 2×10⁵ larvae per liter of liquid nutrient source;
at a temperature of about 20° C. to about 40° C.;
with the liquid nutrient source for about 1, 6, 12, 24 or 48 hours, or about 1 day, 1 week or 1 month; or
a combination of two or more thereof.

15. The method of claim 9, wherein harvesting the larvae comprises:
harvesting mature larvae, or larvae entering the pupa stage of the larva life cycle, or both;
passing the larvae through a filter having a pore size of about 0.3 cm to about 0.5 cm; or
both.

16. An apparatus for removing organic solute from a liquid or for producing larva biomass, comprising:
an enclosed tank, comprising:
a liquid entry port;
a liquid exit port;
a gas entry port;
a gas exit port;
an inner reservoir capable of holding liquid, wherein the liquid entry and exit ports are operably linked to the inner reservoir; and
access means for accessing the inner reservoir of the tank.

17. The apparatus of claim 16, wherein the gas exit port is operably linked to a gas pump.

18. The apparatus of claim 16, further comprising means:
for maintaining fly larva at a depth of about 0.5 to about 3 cm of liquid in the inner reservoir;
for preventing fly larvae from passing through the liquid exit port;
for removing larvae from the inner reservoir;
to monitor organic solute concentration of the liquid in the liquid entry port, the inner reservoir, the liquid exit port or a combination of two of more thereof;
for heating or cooling a liquid in the inner reservoir; or
two or more thereof.

19. The apparatus of claim 16, operably linked to at least one gas trapping scrubber means.

20. The apparatus of claim 16, wherein the inner reservoir contains one or more fly larvae.

21. The apparatus of claim 16, wherein the inner reservoir comprises one or more arrays of modular larvae incubation units.
